(19) United States
(12) Patent Application Publication
(10) Pub. No.: US 2009/0137938 A1
(43) Pub. Date: May 28, 2009

(54) Compressive Garment for Therapeutic Support of the Venous System

(76) Inventor: James Parivash, Los Altos, CA (US)

Correspondence Address: SQUIRE, SANDERS & DEMPSEY L.L.P., 1 MARITIME PLAZA, SUITE 300, SAN FRANCISCO, CA, 94111, US

(21) Appl. No.: 11/946,690

(22) Filed: Nov. 28, 2007

(51) Int. Cl. A61F 13/06; A61B 17/00

(52) U.S. Cl. 602/63; 606/201

(57) ABSTRACT

A compression garment is designed to be worn like a sock to prevent and treat varicose veins. The garment has a resiliency that provides compressive support to the wearers venous structures during periods of raised venous pressure, and has a compliance that allows venous blood to flow back to the heart. Compressive support is provided in either the foot, calf area, or thigh area, or combinations thereof. The garment includes one or more holes to allow the wearer's heel or toes to be exposed outside of the garment such that the wearer is able to dissipate sufficient body heat and remain comfortable during long periods of wearing the garment. The garment can also include a perforated region having a plurality or relatively vent holes.

### BACKGROUND OF THE INVENTION

1. Field of the Invention

This invention relates generally to therapeutic compression garments, more particularly, to compression socks for preventing and treating varicose veins in the lower limbs.

2. Description of the State of the Art

Varicose veins are bulging, sometimes tortuous, elongated veins that usually have a bluish or dark purple color and are commonly found in the legs. It is generally recognized that occupation and posture are major factors leading to varicose veins and contribute significantly to the effects of raised venous pressure and incompetence of primary structures in the vein wall and valves. Persons who stand or are in sedentary positions for long periods of time, such as nursing staff and factory workers, are particularly susceptible to varicose vein development. If not prevented or left untreated, varicose veins can lead to more serious problems, such as thrombophlebitis. This occurs when a blood clot causes inflammation in one or more of veins, typically in the legs. A clot in a deep vein increases the risk of serious health problems, including a dislodged clot traveling to the lungs and blocking an artery, causing a pulmonary embolism. A clot that travels to the coronary arteries or brain can cause a heart attack or stroke.

Compression therapy has been used to prevent and treat varicose veins or varicosities and associated complications, such as thrombophlebitis and venous ulceration. A sock-type orthosis has been used to provide compression to all or part of the leg starting from the ankle, as described in U.S. Pat. No. 6,572,574 to Gardon-Mollard. Generally, compression socks are elasticized stockings worn from the toes to the knee or sometimes to the top of the leg. By increasing venous blood flow to the heart and reducing pressure on the veins, compression socks help reduce the symptoms related to chronic venous insufficiency, such as swelling, discomfort, varicose veins, leg ulcers, sensitive and damaged skin, and skin color changes.

Sock-type compression garments are typically worn as soon as a person gets out of bed, before gravity causes blood to pool in varicose veins. The compression sock is intended to be worn all day, and taken off only when the person is lying down with legs raised at or above the level of the heart. Unlike typical socks, compression socks are commonly made of a thick, durable, closely knit fabric to enable the sock to provide sufficient compression that supports the wearer's venous system throughout the day. In some people, this type of fabric, in combination with the relatively tight fit of compression socks over the entire foot and calf, can overwhelm the wearer's natural heat regulating system. Heat builds up in the leg as well as the rest of the wearer's body. This is aggravated by compression socks that are worn up to the top of the thigh area, by warm climates and by indoor ventilation systems that are set for the comfort of persons not wearing compression garments. It is believed that, because of the compression sock, the wearer is unable to dissipate sufficient body heat or absorbs more heat from surroundings than can be dissipated.

The heat buildup caused by compression socks makes them very uncomfortable to wear for long periods of time. As a result, many wearers do not wear the compression socks all day, or they stop wearing them altogether. This can allow the serious health complications mentioned previously to develop. Therefore, there is a need for a compression sock that provides therapeutic compression and venous support to prevent or treat varicose veins while simultaneously allowing the user's body to sufficient dissipate heat to remain comfortable. The present invention satisfies this and other needs.

### SUMMARY OF THE INVENTION

Briefly and in general terms, the present invention is directed to a compression garment used to prevent and treat varicose veins in the lower limbs, and complications associated with varicose veins.

In an aspect of the present invention, a compression garment comprises a sock including a first opening formed through a top region of the sock and a second opening formed through a bottom region of the sock, the first opening sized to allow a user's foot to pass through the first opening and into the sock, the second opening sized to allow one or both of the user's foot tip and foot heel to pass through the second opening and out of the sock, the sock having a resiliency and a compliance that provide therapeutic support to the user's venous system such that when the sock is worn on a leg of the user, the resiliency of the sock inhibits blood from flowing backward or stagnating in a vein during a period of raised venous pressure, the compliance of the sock allows blood to flow forward in the vein, thereby ensuring adequate venous return.

In another aspect of the present invention, a method of compression therapy comprises wearing a tubular garment that applies compression to at least a portion of a person's leg above the ankle and to at least a portion of a person's foot, the garment having a resiliency and a compliance, using the resiliency of the garment to inhibit blood from flowing backward or stagnating in a vein during a period of raised venous pressure, using the compliance of the garment to allow blood to flow forward in the vein, and exposing one or both of the tip and the heel of the person's foot through an opening formed through the garment.

In a further aspect of the present invention, a compression garment comprises an upper tubular segment including an upper elastic section adapted to provide therapeutic compression to at least a portion of a user's leg when surrounded by the upper elastic section, the therapeutic compression to the leg sufficient to inhibit blood from flowing backward or stagnating in a vein in the leg during a period of raised venous pressure, the therapeutic compression to the leg insufficient to prevent blood from flowing forward in the vein in the leg, and a lower tubular segment attached to the upper tubular segment, the lower tubular segment including either a toe opening and a heel opening, the toe opening sized to allow the user's toes to pass through the toe opening, the heel opening sized to allow the user's heel to pass through the heel opening, the lower tubular segment further including a lower elastic section adapted to provide therapeutic compression to a portion of a user's foot when surrounded by the second elastic section, the therapeutic compression to the foot sufficient to inhibit blood from flowing backward or stagnating in a vein in the foot during the period of raised venous pressure, the therapeutic compression to the foot insufficient to prevent blood from flowing forward in the vein in the foot.

In other aspects of the invention, a compression garment comprises a foot covering including a compressive material having a resiliency that provides therapeutic support to the wearer's venous system such that when the garment is worn, the resiliency of the compressive material inhibits blood from flowing backward or stagnating in a vein covered by the compressive material during a period of raised venous pressure, the foot covering further including a toe region, a sole region, and a heel region, at least one of the toe, sole, and heel regions having a plurality of perforations.

The features and advantages of the invention will be more readily understood from the following detailed description which should be read in conjunction with the accompanying drawings.

### BRIEF DESCRIPTION OF THE DRAWINGS

FIG. 1 is a side view of a compression sock.

FIG. 2 is a perspective view of a compression garment showing the garment in a rest state.

FIG. 3 is a perspective view of the compression garment of FIG. 2 showing the garment in an expanded state over a portion of a leg.

FIG. 4 is a perspective view of a compression garment showing a toe region and a heel region, each having a plurality of perforations.

FIG. 5 is a perspective view of a compression garment showing a perforated sole region.

### DETAILED DESCRIPTION OF THE INVENTION

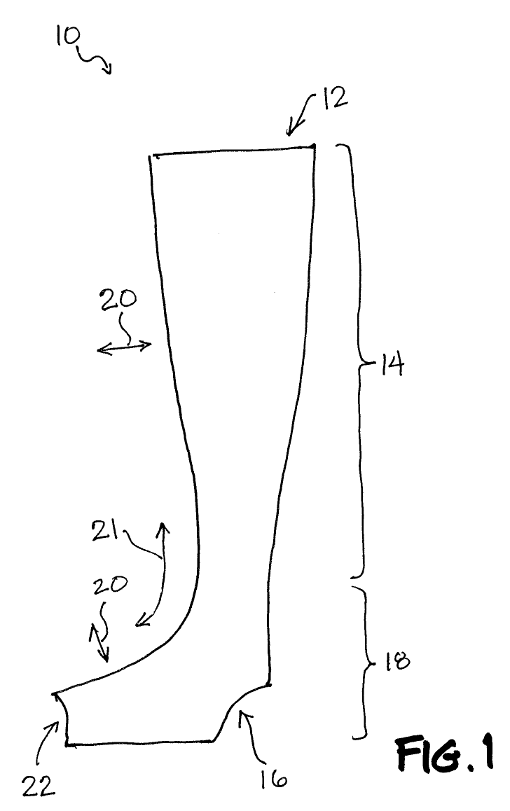

Referring now in more detail to the exemplary drawings for purposes of illustrating embodiments of the invention, wherein like reference numerals designate corresponding or like elements among the several views, there is shown in FIG. 1 a compression sock 10 for preventing and treating varicose veins and associated complications. As used herein, the term sock refers to a stocking-type garment having sufficient length to cover at least a portion of a person's leg, for example a person's foot.

In FIG. 1, the sock 10 includes a first opening 12 formed through a top region 14 of the sock and a second opening 16 formed through a bottom region 18 of the sock. The first opening 12 is sized to allow a user's foot to pass through the first opening and into the sock. The second opening 16 sized to allow the user's foot heel to pass through the second opening and out of the sock. The sock 10 further includes a third opening 22 sized to allow the user's foot tip to pass through the third opening and out of the sock.

Applicant has found that removal of a relatively small amount of sock material to form either one or both of the second or third openings 16, 22 in the bottom region 18 results in an unexpected, disproportionately large increase in comfort that enables a person to wear the sock 10 for long periods of time, without sacrificing or diminishing therapeutic compression in areas that need it. Openings in the bottom region 18 are preferably located at either the heel, toe, or both heel and toe regions of the foot since placing ventilation holes at other parts of the foot (apart from the toes and heel) or above the ankle might reduce overall support of the venous system, diminish therapeutic compression locally at the ventilated areas, and possibly result in varicose vein development at the ventilated areas. A particular material, fabric, or weave pattern that is less insulative or more heat conductive can be used to increase comfort. However, such a material, fabric, or weave is likely to be more costly to manufacture and/or less durable, which requires the wearer to replace the garment more frequently.

A sock without an opening in the bottom region 18 causes an uncomfortable heat buildup in the wearer's body and strains the ability of the wearer to regulate body temperature. It is believed that in some people, a sock without an opening in the bottom region 18 overwhelms the body's natural heat regulating system. With an opening in the bottom region 18, the wearer is able to dissipate enough heat from the wearer's heel or toes to stay comfortable all day while wearing the sock 10. Compression of the heel and toes is believed to be unwarranted since varicose vein development rarely occurs at these parts of the body. Compression of the instep and arch, between the heel and toe, is desirable since varicose vein development can occur in these areas as well as in the calf and thigh areas. Thus, a compression garment that loosely covers the foot, or does not cover the foot at all, will not provide as much venous support as one that provides some compression to at least a portion of the foot.

In other embodiments, the sock 10 has only the first and third openings 12, 22 and does not have the second opening 16. In yet other embodiments, the sock 10 only has the first and second openings 12, 16 and does not have the third opening 22.

Referring again to FIG. 1, the sock 10 is preferably made of an a durable, elastic material that can include, without limitation, nylon fabric, synthetic fibers, natural fibers, and combinations thereof. In this way, the sock 10 is able to stretch from its rest state or unexpanded state, as shown in FIG. 1, to an expanded or stretched state when worn.

The sock 10 has a resiliency and a compliance that provides therapeutic support to the user's venous system such that when the sock is worn on a leg of the user, the resiliency of the sock inhibits blood from flowing backward or stagnating in a vein during a period of raised venous pressure.

The venous system includes, without limitation, the long saphenous veins (LSVs), their tributaries, the short saphenous veins (SSVs), and their tributaries. The LSVs extend up the side of the leg from the foot to the groin, and the SSVs run up the back of the calf from the foot to the knee. The venous walls of the LSVs and SSVs are relatively thin and not supported by the skeletal muscles. To ensure adequate venous return (i.e., forward flow of blood back toward the heart) from the lower limb, the superficial veins, deep veins, perforating veins, bicuspid valves, and the calf muscle must work in concert. A period of raised venous pressure occurs when the wearer is standing, sitting, or in another stationary position in which a leg is below the level of the heart. Calf muscles or other muscles in the leg often do not function to a sufficient degree while a person is in a stationary position, which, in part, results in raised venous pressure. It is believed that in persons who sit or stand for prolonged periods of time, the venous walls become unable to withstand the hydrostatic pressure due to the local high pressure and lack of the pumping action of the leg muscles. Consequently, the venous valves fail to function appropriately and the venous blood gradually accumulates or stagnates in the leg, which eventually results in varicose vein development.

As used herein, resiliency refers to the ability of the sock 10 to contract after being stretched or expanded, or to a tendency to return to a rest state or unexpanded state. Spring-like elements within the sock 10 store energy when the sock 10 stretched in radial directions 20. The spring-like elements apply compressive pressure to the leg and/or foot in the radial directions 20. The spring-like elements can include, without limitation, elastic bands, fibers, or yarns, and can be oriented in straight, woven, knitted, or undulating patterns.

The compliance of the sock 10 allows blood to flow forward in the vein, thereby ensuring adequate venous return. As used herein, compliance refers to the ability of the sock 10 to stretch, expand, or deform in response to some external force, such as when the sock 10 is pulled over a leg. If the sock does not have sufficient resiliency or does not stretch enough, the resilient elements within the sock 10 will apply excessive compressive pressure that blocks blood from flowing forward in the vein and returning to the heart.

In the bottom region 18 of the sock 10, the resiliency of the sock inhibits blood from flowing backward or stagnating in a vein in the user's foot and the compliance of the sock allows blood to flow forward in the vein in the user's foot. In the top region 14 of the sock 10, the resiliency of the sock inhibits blood from flowing backward or stagnating in a vein in the user's calf and the compliance of the sock allows blood to flow forward in the vein in the user's calf.

Still referring to FIG. 1, the sock 10 extends continuously and circumferentially around portions of the leg and foot. In this way, the sock 10 automatically provides the appropriate therapeutic compression, by means of its resiliency and compliance, as soon as the sock 10 is slipped over the foot and leg of a user. Preferably, though not necessarily, the sock 10 has no side opening or other opening extending in a longitudinal direction 21 along the entire length of the sock. If left open, such a large side opening would dimension a garment's ability to provide venous support. A garment with such a side opening does not automatically provide compression since a user is required to manually close the opening, such as with a hook-and-loop device or snaps, to allow the garment to provide some compression.

Further, the sock 10 is unlike existing athletic straps, supports, and braces that a user wraps tightly around a body part. Such athletic support garments are not designed to provide venous support when a person is merely standing, sitting, or in a stationary position. Athletic support garments are designed to support joints or muscles during periods of physical movement and are, therefore, less compliant than therapeutic compressive garments for venous support, are overly compressive, and inhibit venous return to the heart.

A method will now be described in accordance with an embodiment of the present invention. The method includes, wearing a tubular garment that applies compression to at least a portion of a person's leg above the ankle and to at least a portion of a person's foot. The method also includes using the resiliency of the garment to inhibit blood from flowing backward or stagnating in a vein during a period of raised venous pressure, and using the compliance of the garment to allow blood to flow forward in the vein. The method further includes exposing one of the tip and the heel of the person's foot through an opening formed through the garment. Exposing one of the tip and the heel allows the person to better regulate body temperature and, thereby, increase comfort while wearing the garment.

Optionally, using the resiliency includes using the resiliency to inhibit blood from flowing backward or stagnating in a vein in the person's foot, and using the compliance includes using the compliance to allow blood to flow forward in the vein in the foot.

In a further embodiment, using the resiliency includes using the resiliency to inhibit blood from flowing backward or stagnating in a vein in the person's calf region, thigh region, or both the calf and thigh regions.

In a still further embodiment, the method further includes exposing another one of the tip and heel of the person's foot through another opening formed through the garment. Exposing both the foot tip and the heel allows the wearer to regulate body temperature even more and, thereby, increase comfort.

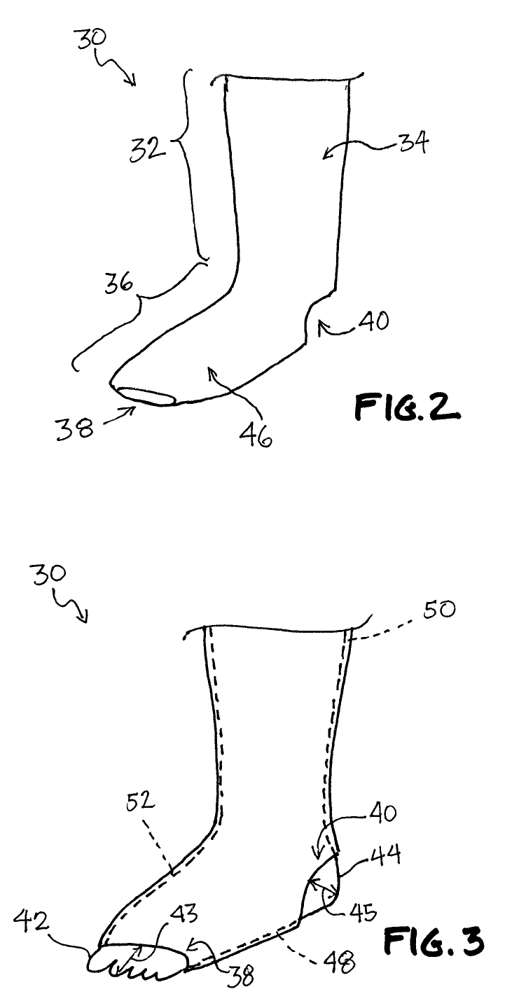

In FIGS. 2 and 3, there is shown compression garment 30 for preventing and treating varicose veins in accordance with an embodiment of the present invention. In FIG. 2, the garment 30 is in its unexpanded or rest state. In FIG. 3, the garment is in its expanded or stretched state. The garment 30 includes an upper tubular segment 32 including an upper elastic section 34 adapted to provide therapeutic compression to at least a portion of a user's leg when surrounded by the upper elastic section. The therapeutic compression to the leg is sufficient to inhibit blood from flowing backward or stagnating in a vein in the leg during a period of raised venous pressure. Further, the therapeutic compression to the leg is insufficient to prevent blood from flowing forward in the vein in the leg. Optionally, the upper elastic section 34 is disposed along the upper tubular segment so as to provide therapeutic compression to either the user's calf 50, thigh, or both calf and thigh.

The garment 30 further includes a lower tubular segment 36 attached to the upper tubular segment 34. The lower tubular segment 36 includes both a toe opening 38 and a heel opening 40. As shown in FIG. 3, the toe opening 38 is sized to allow the user's toes 42 to pass through the toe opening. The heel opening 40 sized to allow the user's heel 44 to pass through the heel opening. The toe and heel openings 38, 40 may stretch to allow the toes or heel to pass through.

In other embodiments, the upper tubular segment 32 extends above the top of the thigh to the waist line, so that the garment 30 is worn like a pair of pants. In further embodiments, the toe opening 38 is configured to allow the toes 42 to extend out of the garment 30 by a distance 43 from about two inches (about five centimeters) to about five inches (about 13 centimeters). In other embodiments, the heel opening 40 is configured to allow the heel 44 to extend out of the garment 30 by a distance 45 from about two inches (about five centimeters) to about four inches (about ten centimeters). In other embodiments, the toe opening 38 or the heel opening 40 allows from about 15% to about 30% of the length of the wearer's foot to be exposed outside of the garment 30.

Referring again to FIGS. 2 and 3, the lower tubular segment 36 of the garment 30 further includes a lower elastic section 46 adapted to provide therapeutic compression to a portion of a user's foot 48 when surrounded by the second elastic section. The therapeutic compression to the foot 48 is sufficient to inhibit blood from flowing backward or stagnating in a vein in the foot during the period of raised venous pressure. Also, the therapeutic compression to the foot is insufficient to prevent blood from flowing forward in the vein in the foot. Optionally, the lower elastic section 46 is disposed along the lower tubular segment so as to provide therapeutic compression to the user's instep 52.

In other embodiments, the lower tubular segment 36 has only one of either the toe opening 38 or the heel opening 40.

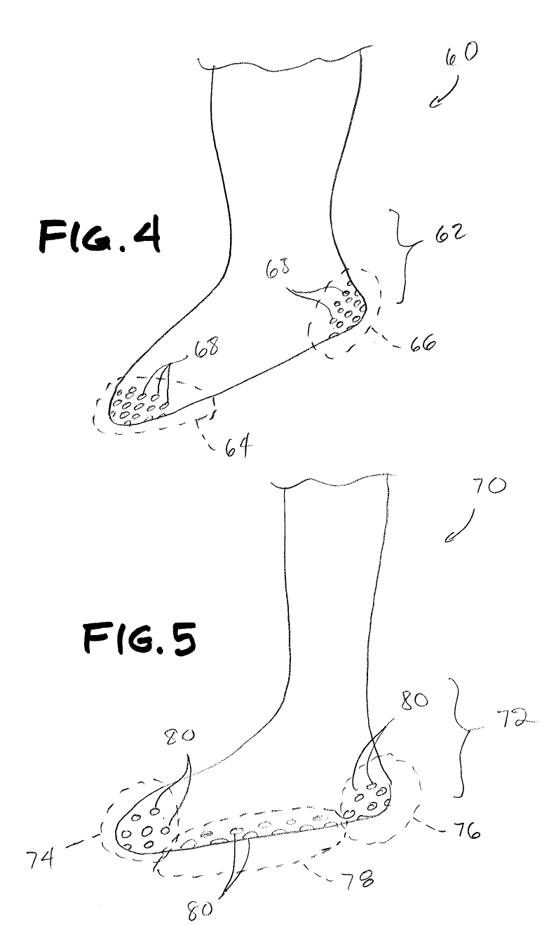

Referring next to FIG. 4, there is shown a compression garment 60 in accordance with an embodiment of the present invention, for preventing and treating varicose veins. The garment 60 comprises a foot covering 62 is made from a compressive material that provides therapeutic compression to areas of the wearer's anatomy that are covered by the garment. The compressive material has a carefully selected resiliency that provides therapeutic support to the wearer's venous system such that when the foot covering 62 is worn, the resiliency inhibits blood from flowing backward or stagnating in a vein covered by the compressive material during a period of raised venous pressure.

The foot covering 62 includes a toe region 64 and a heel region 66 that are located so that they cover the wearer's toes and heel, respectively. The toe and heel regions 64, 66 have a plurality of perforations 68 to provide for ventilation that allows the wearer to dissipate body heat. The perforations can have a diameter from about 0.3 cm (⅛ inch) to about 1.3 cm (½ inch). The perforations can be spaced apart by a distance of about 0.3 cm (⅛ inch) to about 1.3 cm (½ inch), depending on the size of the perforations.

The toe and heel regions 64, 66 can be made of the same material as the remainder of the foot covering 62 or a different material. Other suitable materials include, without limitation, netting and a non-compressive material.

In FIG. 5, there is shown a compression garment 70 in accordance with another embodiment of the present invention. The garment 70 is similar to the garment 60 of FIG. 4 with the addition of a perforated sole region 78. The sole region 78 is located on the foot covering 72 so that the sole region 78 covers the sole or bottom of the wearer's foot. The sole region 78 has a plurality of perforations to provide for ventilation that allows the wearer to dissipate body heat. The perforations 80 can have the same or different diameter as the perforations in the toe and heel regions. The sole region 78 can be made of the same material as the remainder of the foot covering 72 or a different material.

In the illustrated embodiments of FIGS. 4 and 5, the perforations 68, 80 are circular and uniform in size and spacing. The perforations are preferably sized or shaped so that the heel or toes of the user are prevented from passing entirely through the perforations. In this way, the position of the foot covering 62, 72 is less likely to shift on the user's foot. In other embodiments, the perforations 68, 80 can have other shapes, such as slits, elliptical and polygonal. In yet other embodiments, the perforations are not uniform in size or spacing. For example, the perforations at the toe region 64,74 can have a size or spatial distribution that is different than those at the heel region 66, 76. In still other embodiments, only one of the toe region, sole region, or heel region has perforations.

While several particular forms of the invention have been illustrated and described, it will also be apparent that various modifications can be made without departing from the scope of the invention. It is also contemplated that various combinations or subcombinations of the specific features and aspects of the disclosed embodiments can be combined with or substituted for one another in order to form varying modes of the invention. For example, a compressive sock can have a perforated sole region or a perforated heel region combined with a toe opening sized to allow the toes of the wearer to pass through the toe opening. Accordingly, it is not intended that the invention be limited, except as by the appended claims.

What is claimed is:

1. A compression garment for preventing and treating varicose veins, comprising:
a sock including a first opening formed through a top region of the sock and a second opening formed through a bottom region of the sock, the first opening sized to allow a user's foot to pass through the first opening and into the sock, the second opening sized to allow one or both of the user's foot tip and foot heel to pass through the second opening and out of the sock, the sock having a resiliency and a compliance that provide therapeutic support to the user's venous system such that when the garment is worn on a leg of the user, the resiliency of the sock inhibits blood from flowing backward or stagnating in a vein during a period of raised venous pressure, the compliance of the sock allows blood to flow forward in the vein, thereby ensuring adequate venous return.

2. The garment of claim 1, wherein when the garment is worn, the resiliency of the sock inhibits blood from flowing backward or stagnating in a vein in the user's foot and the compliance of the sock allows blood to flow forward in the vein in the user's foot.

3. The garment of claim 1, wherein when the garment is worn, the resiliency of the sock inhibits blood from flowing backward or stagnating in a vein in the user's calf and the compliance of the sock allows blood to flow forward in the vein in the user's calf.

4. The garment of claim 1, wherein the sock further includes a third opening sized to allow the other one of the user's foot tip and the foot heel to pass through the third opening and out of the sock.

5. The garment of claim 1, wherein the sock further includes a perforated region having a plurality of perforations, each perforation sized smaller than the first opening and the second opening, the perforated region located on the sock so that the perforated region covers at least one of the user's foot tip, heel, or sole when the garment is worn.

6. A method of compression therapy to prevent and treat varicose veins, comprising:
wearing a tubular garment that applies compression to at least a portion of a person's leg above the ankle and to at least a portion of a person's foot, the garment having a resiliency and a compliance;
using the resiliency of the garment to inhibit blood from flowing backward or stagnating in a vein during a period of raised venous pressure;
using the compliance of the garment to allow blood to flow forward in the vein; and
exposing one or both of the tip and the heel of the person's foot through an opening formed through the garment.

7. The method of claim 6, wherein using the resiliency includes using the resiliency to inhibit blood from flowing backward or stagnating in a vein in the person's foot, and wherein using the compliance includes using the compliance to allow blood to flow forward in the vein in the foot.

8. The method of claim 6, wherein using the resiliency includes using the resiliency to inhibit blood from flowing backward or stagnating in a vein in the person's calf region, thigh region, or both the calf and thigh regions.

9. The method of claim 6, further comprising exposing another one of the tip and heel of the person's foot through another opening formed through the garment.

10. The method of claim 6, further comprising ventilating another one of the tip and heel of the person's foot through a plurality of perforations formed in the garment.

11. A compression garment for preventing and treating varicose veins, comprising:
an upper tubular segment including an upper elastic section adapted to provide therapeutic compression to at least a portion of a user's leg when surrounded by the upper elastic section, the therapeutic compression to the leg sufficient to inhibit blood from flowing backward or stagnating in a vein in the leg during a period of raised venous pressure, the therapeutic compression to the leg insufficient to prevent blood from flowing forward in the vein in the leg; and
a lower tubular segment attached to the upper tubular segment, the lower tubular segment including either a toe opening or a heel opening, the toe opening sized to allow the user's toes to pass through the toe opening, the heel opening sized to allow the user's heel to pass through the heel opening, the lower tubular segment further including a lower elastic section adapted to provide therapeutic compression to a portion of a user's foot when surrounded by the second elastic section, the therapeutic compression to the foot sufficient to inhibit blood from flowing backward or stagnating in a vein in the foot during the period of raised venous pressure, the therapeutic compression to the foot insufficient to prevent blood from flowing forward in the vein in the foot.

12. The garment of claim 11, wherein the upper elastic section is disposed along the upper tubular segment so as to provide therapeutic compression to either the user's calf, thigh, or both calf and thigh.

13. The garment of claim 11, wherein the lower elastic section is disposed along the lower tubular segment so as to provide therapeutic compression to the user's instep.

14. The garment of claim 11, wherein the lower tubular segment includes both the toe opening and the heel opening.

15. The garment of claim 11, wherein the lower tubular segment includes a perforated portion having a plurality of perforations sized smaller than either the toe opening or the heel opening, the perforated portion located on the lower tubular segment so as to be at or adjacent at least one of the user's toes, sole, and heel when the garment is worn.

16. A compression garment for preventing and treating varicose veins, comprising: a foot covering including a compressive material having a resiliency that provides therapeutic support to the wearer's venous system such that when the garment is worn, the resiliency of the compressive material inhibits blood from flowing backward or stagnating in a vein covered by the compressive material during a period of raised venous pressure, the foot covering further including a toe region, a sole region, and a heel region, at least one of the toe, sole, and heel regions having a plurality of perforations.

17. The garment of claim 16, wherein the perforations have a diameter from about 0.3 cm (⅛ inch) to about 1.3 cm (½ inch).

18. The garment of claim 16, wherein an opening is formed in one of the sole region and the heel region, the opening sized to allow the wearer's foot tip or foot heel to pass through the opening and out of the garment.

19. The garment of claim 16, wherein the compressive material has sufficient compliance to allow blood to flow forward in the vein covered by the compressive material when the garment is worn.
